(19) United States
(12) Patent Application Publication
(10) Pub. No.: US 2010/0198084 A1
(43) Pub. Date: Aug. 5, 2010

(54) SENSOR COMPRISING A MATERIAL WHICH GENERATES AN ELECTRICAL SIGNAL IN RESPONSE TO ELONGATION

(76) Inventors: Kap Jin Kim, Gyeonggi-do (KR); Hyun Jeong Kim, Seoul (KR)

Correspondence Address: ROBERT E. BUSHNELL & LAW FIRM, 2029 K STREET NW, SUITE 600, WASHINGTON, DC, 20006-1004, US

(21) Appl. No.: 12/733,816

(22) PCT Filed: Sep. 22, 2008
(86) PCT No.: PCT/KR2008/005628

§ 371 Date: Apr. 1, 2010

(30) Foreign Application Priority Data
Sep. 12, 2007 (KR) 10-2007-0096409
Sep. 21, 2008 (KR) 10-2008-0092470

(51) Int. Cl. A61B 5/02; A61B 5/08

(52) U.S. Cl. 600/484; 600/503

(57) ABSTRACT

The present invention relates to a sensor comprising a material which generates an electrical signal in response to elongation. More particularly, the present invention is directed to an apparatus for measuring a length or volume change, which comprises a sensor comprising a piezoelectric material which generates an electrical signal in response to elongation.

### TECHNICAL FIELD

The present invention relates to a sensor comprising a material which generates an electrical signal in response to elongation. More particularly, the present invention is directed to an apparatus for measuring a length or volume change, which comprises a sensor comprising a piezoelectric material which generates an electrical signal in response to elongation.

### BACKGROUND ART

A respiratory rate and a heart rate are basic indicators which indicate a health condition of mammals including human, and a pulse wave velocity (PWV) which is calculated from the measurement of pulse waves is a good indicator for evaluation of elasticity and blockage of arteries.

The respiratory rate is generally defined as the number of movements indicative of inspiration and expiration per minute when a living being is at rest. The respiratory rate can be determined by measuring the number of times the chest and abdomen expand and contract per minute since the chest and abdomen expand and contract one time respectively while breathing one time.

The heart rate is measured by counting the number of pulse waves per minute, which are caused by the expansion and contraction of the arteries responding to the relaxation and contraction of the heart. The heart rate can be measured by counting the number of pulsations per minute generated by the expansion and contraction of the arteries, in response to the heartbeat, which can be palpated at the left chest, head, wrist and ankle.

The pulse delay time, a period of time for which an artery's pulsation generated from a heartbeat reaches the head, wrist, ankle, etc., are different from one another. The pulse wave velocity can be calculated from the lengths of arteries from the heart to the head, wrist, ankle, etc., respectively. There is a significant difference in PWV between a healthy person and a person with arteriosclerosis whose arteries are partially blocked. The smaller the internal diameter of an artery due to the blockage of the artery, the faster the pulse wave velocity. The elasticity and blockage of arteries from the heart to the head, to the wrist, and to the ankle, respective, can be monitored by measuring periodically the pulse wave velocity at certain locations of the body.

While breathing, mainly the circumferences of the chest and abdomen change with time, and while the heart beats, the circumferences of the head, wrist, ankle, etc. are periodically change with time due to the expansion and contraction of arteries. The amplitude of change of the circumferences of the head, wrist, ankle, etc. caused by heartbeats is much smaller than that of the chest and abdomen caused by respiration, whereas the frequency of the change of the circumferences of the head, wrist, ankle, etc. is much higher than that of the chest and abdomen and changes periodically with time.

Equipments for continuously monitoring the heart rate and respiratory rate of patients who are being operated in the hospital, patients with a cardiovascular disease whose conditions of heartbeat and respiration should be observed continuously, and elderly persons who are physically disordered, have been used. According to recent developments in mobile technologies, portable electrocardiogram-respiration signal measuring devices for patients who are likely to die suddenly by myocardial infarction, coronary artery disease, arrhythmia, etc., have been continuously developed and miniaturized. Therefore, such devices can be easily carried and battery consumption thereof is being significantly reduced.

End-tidal carbon dioxide (ETCO₂) measuring device, a respiration measurement device which is presently used mainly for patients who are being operated in hospital, monitors the concentration of carbon dioxide in respiratory gases and calculate the number of respiration during the last 15 to 30 seconds and thereby obtain the respiratory rate. It is advantageous that this method measures accurately the amount of carbon dioxide in the respiratory gases such that the present metabolic condition of the patients can be known as well as the respiratory rate.

However, this method gives the patients pains since a tube must be inserted through the nasal cavity into the bronchus during collecting the respiratory gases in order to measure an accurate amount of carbon dioxide in the exhaled air. Therefore, the use of the ETCO₂ measuring device is restricted to critically ill patients who are under general anesthesia during operation or unconscious. Moreover, there is a disadvantage that the reliability of such ETCO₂ measuring devices is highly dependent on a type of the respiratory gas collection device.

Consequently, various methods for removing from the patients the pains caused by the ETCO₂ measuring device have been studied in order to use the ETCO₂ measuring device for mild patients.

As one method for dealing with the above-mentioned problem of the conventional ETCO₂ measuring device, there is phonocardiography, utilizing the principle of a stethoscope, that measures the respiratory rate by converting sound waves in the airway which are generated during respiration into electrical signals. However, this method does not work well since a phonocardiograph is very sensitive to surrounding noises and therefore the surrounding noises must be lower than heart sounds.

Alternatively, there is another method that measures respiratory signals from electrocardiogram (ECG) signals by using a low frequency filter. However, it is difficult to use practically this method since the intensity of the ECG signals is very low and the error range of the ECG signals is wide.

Besides, there is still another method that obtains respiratory signals. In this method, a belt containing dual coil is tied around the chest and abdomen, and thereby measuring the changes of the inductance or electric capacity of dual coil, which is caused by the physical changes, i.e. the expansion and contraction, of the chest while breathing. However, the dual coil is easily affected by external electromagnetic interference (EMI) and thus an additional sensor for eliminating the influence of the external EMI is needed.

Therefore, it has been required to develop a sensor for measuring the heart rate and respiratory rate, which is not easily affected by the external EMI, is easily wearable and thus is not uncomfortable for patients, can be produced at a low cost, and is more accurate than the conventional devices.

### DISCLOSURE

### [Technical Problem]

The primary object of the present invention is to provide a sensor for measuring change of length or volume, which comprises a material which generates an electrical signal in response to elongation.

Another object of the present invention is to provide an apparatus for measuring a length or volume change, which comprises a sensor comprising a material which generates an electrical signal in response to elongation, wherein said sensor is mounted on or around an object of measurement, and the length or volume change of the object is measured from an electrical signal in response to elongational deformation of the material due to the increase of length or circumference of the object.

Yet another object of the present invention is to provide an apparatus for measuring respiratory rate or heart rate, which comprises a sensor comprising a material which generates an electrical signal in response to elongation, wherein said sensor is mounted on or around a living being of measurement, and the respiratory rate or heart rate of the living being is measured from an electrical signal in response to elongational deformation of the material due to the increase of circumference of the living being.

Still another object of the present invention is to provide an elastic band-type apparatus for measuring respiratory rate or heart rate, which comprises a material layer which generates an electrical signal in response to elongation; two electrode layers, each of which covers respectively either side of said material layer; and an elastic band which wraps said material layer and said two electrode layers.

Further another object of the present invention is to provide a system for measuring respiratory rate or heart rate, which comprises: a sensing unit comprising a sensor which generates an electrical signal in response to elongation; an analog signal processing unit which removes noises from the analog signals measured from said sensor and amplifies noise-removed analog signals; an analog-digital converting unit which converts said noise-removed analog signals into digital signals; a digital signal processing unit which analyzes the digital signals from said analog-digital converting unit and thereby calculate a respiratory rate or heart rate; and a display unit which displays the data of the respiratory rate or heart rate from said digital signal processing unit.

### [Technical Solution]

The above-mentioned primary object of the present invention can be achieved by providing a sensor for measuring change of length or volume, which comprises a material which generates an electrical signal in response to elongation.

The material used for the sensor of the present invention is preferably a piezoelectric polymer. The term “piezoelectricity” as used herein refers to the ability of some materials to generate an electric signal (electric potential or current) in response to applied mechanical stress.

Preferably, the piezoelectric polymer is selected from poly(vinylidene fluoride), poly(vinylidene fluoride) blend, vinylidene fluoride copolymer or nylon-11, etc.

Furthermore, the poly(vinylidene fluoride) blend is preferably selected from a poly(vinylidene fluoride)/poly(methyl methacrylate) blend, a poly(vinylidene fluoride)/poly(vinyl acetate) blend, a poly(vinylidene fluoride)/poly(vinyl acetate) copolymer blend, etc.

In addition, the vinylidene fluoride copolymer is selected from poly(vinylidene fluoride-co-trifluoroethylene), poly(vinylidene fluoride-co-tetrafluoroethylene), poly(vinylidene fluoride-co-hexafluoroethylene), poly(vinylidene fluoride-co-trichlorofluoroethylene), etc.

The piezoelectric polymer, which may be preferably used for the sensor of the present invention, is in the form selected from a film, a sheet, a cylinder, a string, a strand, a fiber, a woven fabric, a nanofiber web, etc.

The above-mentioned another object of the present invention can be achieved by providing an apparatus for measuring a length or volume change, which comprises a sensor comprising a material which generates an electrical signal in response to elongation, wherein said sensor is mounted on or around an object of measurement, and the length or volume change of the object is measured from an electrical signal in response to elongational deformation of the material due to the increase of length or circumference of the object.

Structures constructed with concretes, steel, etc., for example, architectural structures such as buildings and houses, civil engineering structures such as bridges, overpasses and dams, ship structures such as oil tankers and cargo ships, vehicles such as cars and trains, may be equipped with the apparatus for measuring a length or volume change at the part in which it is required to measure the change of length of wire rods due to bending, etc., or the change of volume due to the change of circumference of the structure, and thereby the change of length or volume of the part can be obtained. With this, the safety of the structures can be continuously monitored.

Besides the structures mentioned above, the apparatus for measuring a length or volume change of the present invention may be used to parts which is required to measure the change of length of volume thereof.

The sensor of the present invention may also be applied to animals such as livestocks and wild animals, and plants such as trees so as to measure the change of length, height or circumference of the animal body or the plant trunk. Accordingly, the growth rate of the animals and plants can be known.

The material used for the apparatus for measuring a length or volume change of the present invention is preferably a piezoelectric polymer. In addition, the piezoelectric polymer is preferably selected from poly(vinylidene fluoride), poly(vinylidene fluoride) blend, vinylidene fluoride copolymer or nylon-11, etc.

Furthermore, the poly(vinylidene fluoride) blend is preferably selected from a poly(vinylidene fluoride)/poly(methyl methacrylate) blend, a poly(vinylidene fluoride)/poly(vinyl acetate) blend, a poly(vinylidene fluoride)/poly(vinyl acetate) copolymer blend, etc.

In addition, the vinylidene fluoride copolymer is selected from poly(vinylidene fluoride-co-trifluoroethylene), poly(vinylidene fluoride-co-tetrafluoroethylene), poly(vinylidene fluoride-co-hexafluoroethylene), poly(vinylidene fluoride-co-trichlorofluoroethylene), etc.

The piezoelectric polymer, which may be preferably used for the sensor of the present invention, is in the form selected from a film, a sheet, a cylinder, a string, a strand, a fiber, a woven fabric, a nanofiber web, etc.

The above-mentioned yet another object of the present invention can be achieved by providing an apparatus for measuring respiratory rate or heart rate, which comprises a sensor comprising a material which generates an electrical signal in response to elongation, wherein said sensor is mounted on or around a living being of measurement, and the respiratory rate or heart rate of the living being is measured from an electrical signal in response to elongational deformation of the material due to the increase of circumference of the living being.

The sensor used for the apparatus for measuring respiratory rate or heart rate of the present invention deforms elonagationally in response to the waves generated by the heartbeat, or the change of circumference of animals due to respiration. Therefore, the apparatus for measuring respiratory rate or heart rate of the present invention may be effectively used to measure the respiratory rate or heart rate of the animals through the electrical signals generated by the elongational deformation of the sensor of the present invention.

The material used for the apparatus for measuring respiratory rate or heart rate of the present invention is preferably a piezoelectric polymer. In addition, the piezoelectric polymer is preferably selected from poly(vinylidene fluoride), poly(vinylidene fluoride) blend, vinylidene fluoride copolymer or nylon-11, etc.

Furthermore, the poly(vinylidene fluoride) blend is preferably selected from a poly(vinylidene fluoride)/poly(methyl methacrylate) blend, a poly(vinylidene fluoride)/poly(vinyl acetate) blend, a poly(vinylidene fluoride)/poly(vinyl acetate) copolymer blend, etc.

In addition, the vinylidene fluoride copolymer is selected from poly(vinylidene fluoride-co-trifluoroethylene), poly(vinylidene fluoride-co-tetrafluoroethylene), poly(vinylidene fluoride-co-hexafluoroethylene), poly(vinylidene fluoride-co-trichlorofluoroethylene), etc.

The piezoelectric polymer, which may be preferably used for the sensor of the present invention, is in the form selected from a film, a sheet, a cylinder, a string, a strand, a fiber, a woven fabric, a nanofiber web, etc.

The above-mentioned still another object of the present invention can be achieved by providing an elastic band-type apparatus for measuring respiratory rate or heart rate, which comprises a material layer which generates an electrical signal in response to elongation; two electrode layers, each of which covers respectively either side of said material layer; and an elastic band which wraps said material layer and said two electrode layers.

The material used for the elastic band-type apparatus for measuring respiratory rate or heart rate of the present invention is preferably a piezoelectric polymer. In addition, the piezoelectric polymer is preferably selected from poly(vinylidene fluoride), poly(vinylidene fluoride) blend, vinylidene fluoride copolymer or nylon-11, etc.

Furthermore, the poly(vinylidene fluoride) blend is preferably selected from a poly(vinylidene fluoride)/poly(methyl methacrylate) blend, a poly(vinylidene fluoride)/poly(vinyl acetate) blend, a poly(vinylidene fluoride)/poly(vinyl acetate) copolymer blend, etc.

In addition, the vinylidene fluoride copolymer is selected from poly(vinylidene fluoride-co-trifluoroethylene), poly(vinylidene fluoride-co-tetrafluoroethylene), poly(vinylidene fluoride-co-hexafluoroethylene), poly(vinylidene fluoride-co-trichlorofluoroethylene), etc.

The piezoelectric polymer, which may be preferably used for the elastic band-type apparatus for measuring respiratory rate or heart rate of the present invention, is in the form selected from a film, a sheet, a cylinder, a string, a strand, a fiber, a woven fabric, a nanofiber web, etc.

The two electrode layers of the elastic band-type apparatus for measuring respiratory rate or heart rate of the present invention are selected from Au, Ag, Cu, Pt, Al, Ni or Co, etc. Further, the conductive leads are connected to the two electrode layers.

Preferably, the at least one outer side of said two electrode layers is coated with a polymeric material. The polymeric material is preferably at least one selected from butadiene rubber or latex, isoprene rubber or latex, chloroprene rubber or latex, nitrile rubber or latex, silicon rubber or latex, polyurethane rubber or latex, polyethylene, polyester, polyacryl, polyimide, or polyacetate, etc. In addition, the coating thickness of the polymeric material is preferably between 100 μm and 5 mm.

The elastic band may be produced from the conventional elastic fibers, and the tensile strain of the elastic band is preferably between 0.1 and 0.4.

The above-mentioned further another object of the present invention can be achieved by providing a system for measuring respiratory rate or heart rate, which comprises: a sensing unit comprising a sensor which generates an electrical signal in response to elongation; an analog signal processing unit which removes noises from the analog signals measured from said sensor and amplifies noise-removed analog signals; an analog-digital converting unit which converts said noise-removed analog signals into digital signals; a digital signal processing unit which analyzes the digital signals from said analog-digital converting unit and thereby calculate a respiratory rate or heart rate; and a display unit which displays the data of the respiratory rate or heart rate from said digital signal processing unit.

The system for measuring respiratory rate or heart rate of the present invention may further comprise an auxiliary memory unit. The patient's condition may be checked through the data of the respiratory rate and heart rate of a patient, which are stored in the auxiliary memory unit.

### [Advantageous Effects]

The sensor of the present invention makes it easy to measure the change of length or circumference of various structures, animals and plants.

In addition, the apparatus for measuring respiratory rate or heart rate of the present invention does not give a patient uncomfortable feeling and can be easily wearable. Also, the apparatus for measuring respiratory rate or heart rate is very sensitive to physical changes and therefore can accurately measure the respiratory rate or heart rate.

### DESCRIPTION OF DRAWINGS

FIG. 1 shows the principle of piezoelectricity.

FIG. 2 illustrates an embodiment of the sensor comprising piezoelectric polymer of the present invention.

FIG. 3 is an image of the elastic band-type apparatus which was manufactured by inserting the sensor of FIG. 2 between two elastic bands, and a cross-sectional view of a wrist around which the elastic band-type apparatus is tied.

FIG. 4 is a configuration of the system for measuring respiratory rate or heart rate of the present invention.

FIG. 5 to FIG. 7 are an input buffer circuit (FIG. 5), a filtering circuit (FIG. 6), and an amplication and output circuit (FIG. 7), which are used for the signal processing unit of the system for measuring respiratory rate or heart rate of the present invention.

FIG. 8 shows (a) time domain signal and (b) frequency domain signal when the elastic band-type apparatus for measuring respiratory rate or heart rate of the present invention was worn on the right wrist of a human at rest.

FIG. 9 shows (a) time domain signal and (b) frequency domain signal when the elastic band-type apparatus for measuring respiratory rate or heart rate of the present invention was worn on the chest of a human at rest.

FIG. 10 shows the respiration signals measured on the human chest around which the elastic band-type apparatus for measuring respiratory rate or heart rate of the present invention is tied, for various thickness of the coated silicon rubber.

FIG. 11 is a plot of heartbeat response output signal versus the tensile strain of the elastic band-type apparatus for measuring respiratory rate or heart rate of the present invention worn on the wrist.

FIG. 12 is a graph of pulse wave signals measured at different body locations.

### BEST MODE

Hereinafter, the present invention will be described in greater detail with reference to the following examples and drawings. The examples and drawings are given only for illustration of the present invention and not to be limiting the present invention.

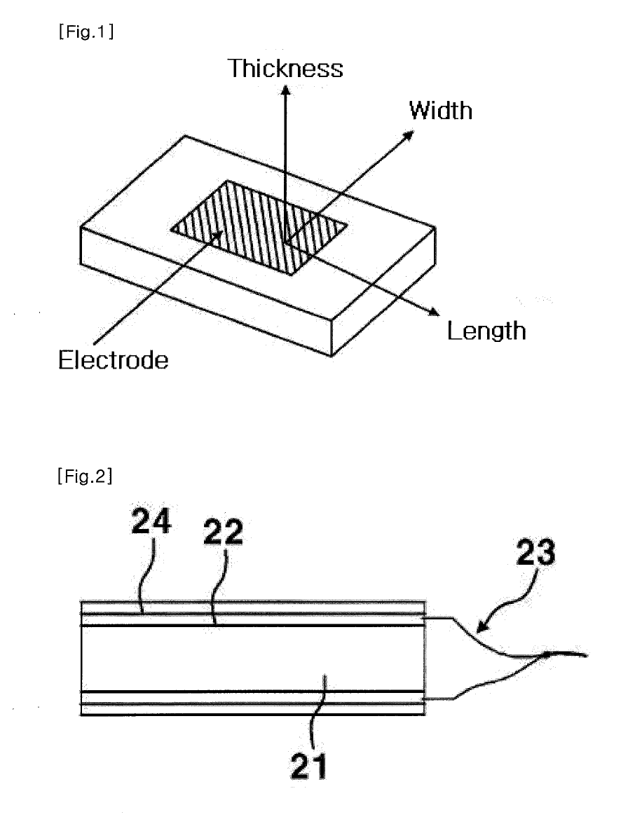

FIG. 1 shows the principle of piezoelectricity of the sensor according to the present invention. When the sensor is pressurized in the direction of thickness, the thickness of the sensor decreases so as to the change of the charge density of the sensor and thereby currents flow (or voltage is induced) in the direction of thickness. The magnitude of the currents (or voltage) is proportional to that of the applied pressure. In addition to the pressurization in the direction of thickness, when the sensor is pressurized or elongated in the direction of length or width, the thickness of the sensor also changes proportionally to the magnitude of the applied pressure or the elongation, which induces currents (or voltage) in the direction of thickness, of which magnitude is proportional to the magnitude of the applied pressure or the elongation.

FIG. 2 illustrates one embodiment of the sensor comprising piezoelectric polymer of the present invention. Referring to FIG. 2, the sensor comprises a piezoelectric polymer film 21, electrode layers 22 which cover respectively both side of the piezoelectric polymer film 21, conductive leads 23 which are connected to the electrode layers by metal rivets, and rubber layers 24 which cover the electrode layers.

The piezoelectric polymer film 21 is produced by the uniaxial or biaxial elongation of the piezoelectric polymer film which is manufactured by the solution casting or melt forming of piezoelectric polymer. The thickness of the piezoelectric polymer film is preferably 6 μm to 2,000 μm. The piezoelectric polymer is preferably selected from poly(vinylidene fluoride), poly(vinylidene fluoride) blend, vinylidene fluoride copolymer or nylon-11, etc.

Furthermore, the poly(vinylidene fluoride) blend is preferably selected from a poly(vinylidene fluoride)/poly(methyl methacrylate) blend, a poly(vinylidene fluoride)/poly(vinyl acetate) blend, a poly(vinylidene fluoride)/poly(vinyl acetate) copolymer blend, etc.

In addition, the vinylidene fluoride copolymer is selected from poly(vinylidene fluoride-co-trifluoroethylene), poly(vinylidene fluoride-co-tetrafluoroethylene), poly(vinylidene fluoride-co-hexafluoroethylene), poly(vinylidene fluoride-co-trichlorofluoroethylene), etc.

The elongated piezoelectric film is corona poled by applying a high electric potential in the direction of thickness. After the corona poling, the dipoles which are randomly oriented in the elongated piezoelectric film, are aligned along the direction of the external electric potential. Then, the corona-poled piezoelectric film has a remanent polarization (Pᵣ) since the dipoles aligned along the direction of the external electric potential do not restore their random orientation after the external electric potential has been removed.

The conductive electrodes are attached to both side of the poled piezoelectric film and thus the conductive electrode layers 22 are produced in the form of a capacitor. The electrode layers may be formed by the application of silver paste on the piezoelectric film. Also, gold (Au), platinum (Pt), silver (Ag), copper (Cu), aluminium (Al), nickel (Ni), cobalt (Co), etc. may be deposited on the piezoelectric film through the methods of sputtering, vacuum thermal deposition, e-beam evaporation, etc. Preferably, the length and width of the piezoelectric film to which the electrode layers are attached are 5 mm to 300 mm and 5 mm to 25 mm, respectively.

Then, the conductive leads 23 may be connected to the upper and lower electrodes of the piezoelectric film which has been coated with the electrode layers and cut to the desired size, by using metal rivets, and small connectors and plugs may be connected to the end of the leads in order to facilitate the connection of the sensor of the present invention to a signal processing unit.

If required, polymeric coatings of butadiene rubber or latex, isoprene rubber or latex, chloroprene rubber or latex, nitrile rubber or latex, silicon rubber or latex, polyurethane rubber or latex, polyethylene, polyester, polyacryl, polyimide, or polyacetate, etc., may be formed through solution casting on the piezoelectric film to which the electrode layers are attached in order for the prevention of abrasion of the electrode layers. Also, the piezoelectric film to which the electrode layers are attached may be laminated with a polymeric film of polyethylene, polyacryl, polyimide, polyacetate, etc. Preferably, the thickness of the coating is 3 mm to 30 mm and that of the lamination is 50 mm to 500 mm.

It is not easy to measure the change of current or electrical potential in response to the respiration or heartbeat since the thickness change of the piezoelectric polymer film, in response to the respiration or heartbeat, is negligible even if the piezoelectric polymer film of the present invention is tied around the chest, abdomen, head, wrist and ankle of a mammal.

On the contrary, when the piezoelectric polymer film of the present invention is tied around the chest, abdomen, head, wrist and ankle, the circumference of the piezoelectric polymer film in response to the respiration or heartbeat changes easily in the direction of length, and thus it is easy to measure the change of current or electrical potential of the piezoelectric polymer film in response to the respiration or heartbeat.

However, it is not easy to fix the piezoelectric polymer film to the chest, abdomen, head, wrist and ankle since the tensile strain of the piezoelectric polymer film is very small. Therefore, it is preferable to tie around the chest, abdomen, head, wrist and ankle by means of an elastic belt which is made of two elastic bands and the piezoelectric polymer film imbedded therebetween. Then, the length of the elastic belt is changed by the circumference change of the chest, abdomen, head, wrist and ankle during respiration or heartbeat. As a result, the respiration rate or heart rate can be measured by the length change of the piezoelectric polymer film which is imbedded between the elastic bands.

Meanwhile, when the piezoelectric polymer film is coated with a protective layer, the adhesion between the electrode layer and the elastic band is bad and thus there occurs the slippage between the elastic band and the electrode layer. Therefore, the current (or electrical potential) signals generated from the piezoelectric polymer film are weak since the piezoelectric polymer film does not elongate together with the elongation of the elastic band due to the slippage. Consequently, it is difficult to monitor the heartbeat and respiration. Moreover, the piezoelectric polymer film with low flexural rigidity wrinkles in the process of returning to its original length when removing the elastic band-type apparatus from the body.

The piezoelectric polymer film which is laminated with a polymer film such as a polyester film has high flexural rigidity and thus the occurrence of the wrinkles in the piezoelectric polymer film can be avoided in the process of returning to the original length. However, the piezoelectric polymer film laminated with a polymer film such as a polyester film also has high modulus of elasticity and, therefore, the amound of the lengthwise elongation of the piezoelectric polymer film, which results from the increase of the circumference of the chest, head, wrist, ankle, etc. in response to the heartbeat and respiration, is smaller than the piezoelectric polymer film coated with the protective layer. Accordingly, the gain of the amplifier should be highly increased. In this case, however, noise signals are also amplified and, therefore, it is difficult to isolate the pure heartbeat and respiration signals from the noise signals without a very efficient filter circuit.

In order to solve the above-mentioned problems, the present inventors has coated with silicon-based rubber solution, e.g. polydimethylsiloxane (PDMS) solution, either or both sides of a piezoelectric polymer film only with two electrode layers, a piezoelectric polymer film with two electrode layers each of which is coated with a electrode layer and then coated with a polymer film, and a piezoelectric polymer film each of which is coated with a electrode layer and then laminated with a polymer film, to a preferable thickness of 100 μm to 5 mm.

As a result, when the elastic band has been elongated, the friction coefficient of the coated surface of the piezoelectric polymer film with the electrode layers increased and, therefore, the slippage of the piezoelectric polymer film decreased and the flexural rigidity of the piezoelectric polymer significantly increased. Thus, the occurrence of wrinkles can also be avoided simultaneously in the process of returning to the original length when removing the elastic band from the human body. The elastic band may be made of the conventional elastic fibers, but not limited thereto.

An elastic band-type apparatus for measuring respiratory rate or heart rate as a preferable embodiment of the present invention, may be manufactured as follows. A piezoelectric polymer film to which two electrode layers are attached respectively on either side thereof is positioned between a first elastic band and a second elastic band which are wider and longer than the piezoelectric polymer film (in this case, the width of the first elastic band is equal to that of the second elastic band and the lengths of the elastic bands should be longer than that of the film-type condenser). The two elastic bands are sewed together so as to allow the piezoelastic polymer film with two electrode layers to adhere closely to the two elastic bands. A connector or a plug which are connected to the two electrode layers is preferably fixed at the elastic bands.

It may be made easy to mounting or removal of the elastic band-type apparatus by attaching a pair of velcro tape (hook and fastener tape) or a pair of plastic buckle to the elastic bands.

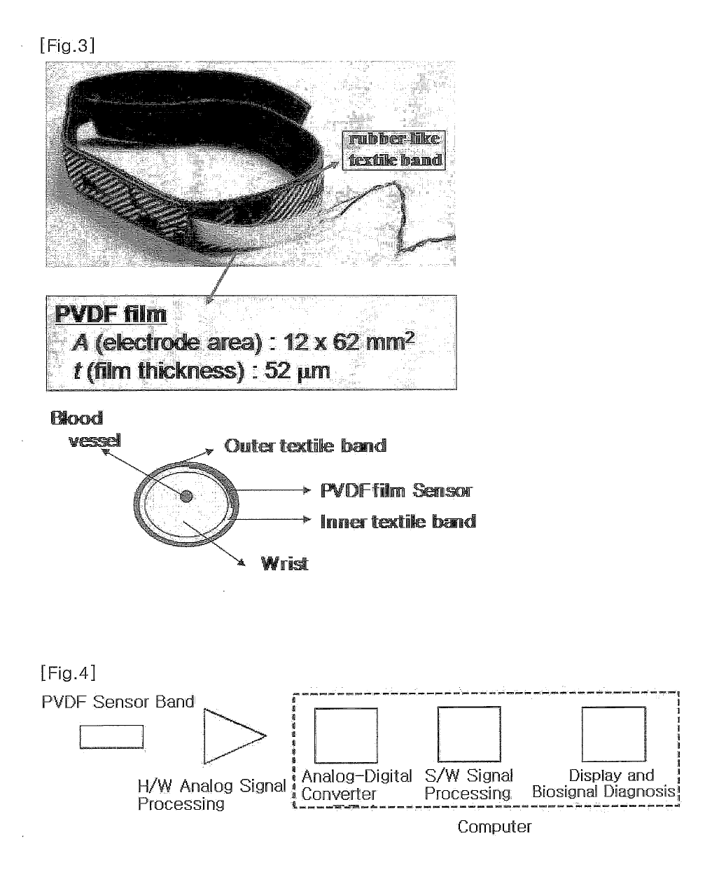

FIG. 3 is an image of the elastic band-type apparatus which was manufactured by inserting the sensor of FIG. 2 between two elastic bands, and a cross-sectional view of a wrist around which the elastic band-type apparatus is tied.

A system for measuring respiratory rate or heart rate may be manufactured by means of the elastic band-type apparatus of the present invention. One embodiment of the system for measuring respiratory rate or heart rate of the present invention is illustrated in FIG. 4.

Referring to FIG. 4, the system for measuring respiratory rate or heart rate of the present invention comprises a sensing unit, an analog signal processing unit, an analog-digital converting unit, a digital signal processing unit and a display unit. The sensing unit, analog signal processing unit and display unit may be embodied as a computer. The sensing unit is an elastic band-type apparatus imbedded with a piezoelectric polymer film.

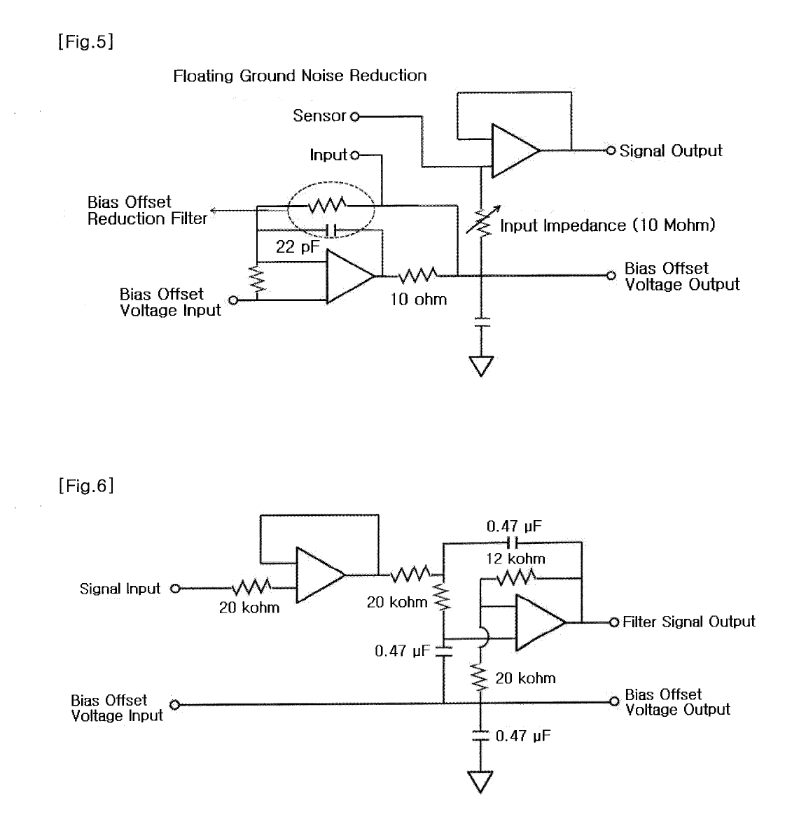

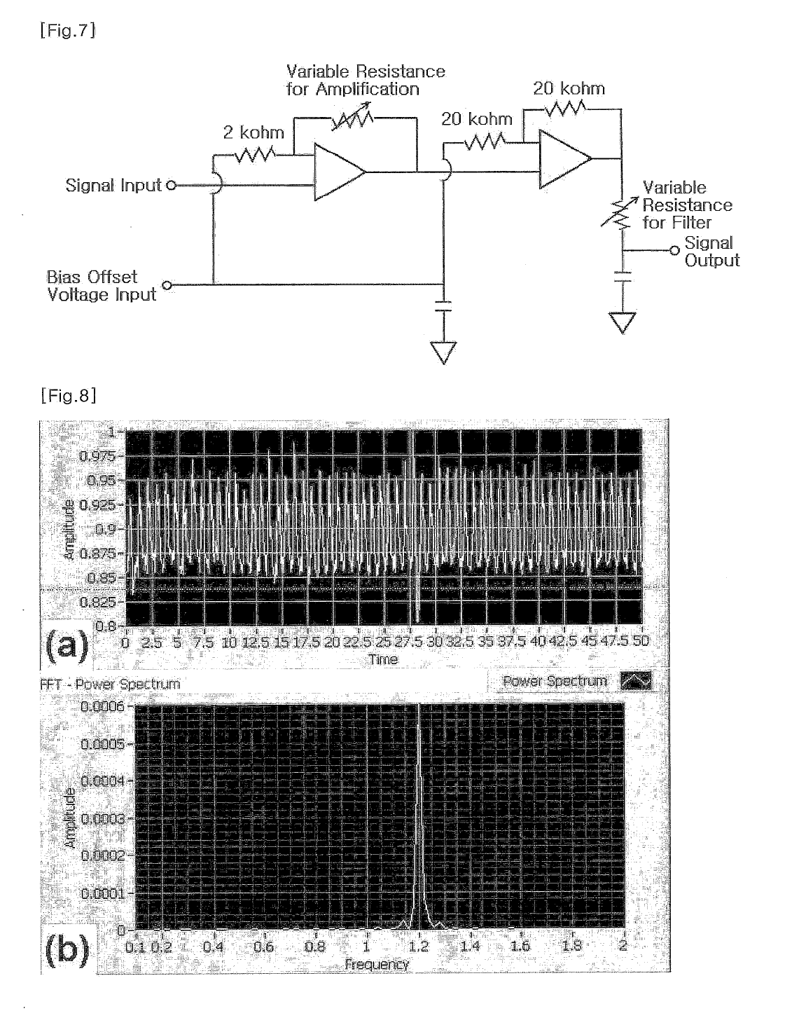

The analog signal processing unit may be made by a known method and, specifically, may be made up of an input buffer circuit (FIG. 5) which is connected to the sensing unit and receives electrical signals measured from the sensing unit, a filtering circuit (FIG. 6), and an amplication and output circuit (FIG. 7) which comprise an amplication circuit for amplifying the signals.

The analog-digital converting unit, digital signal processing unit and display unit may be conventional apparatuses.

Analog voltage signals from the analog signal processing unit (ASP unit) are converted to digital signals by means of an analog-digital converter (ADC) in a data acquisition board (DAQ board), and the data obtained by analyzing the digital signals by means of a software, such as LabVIEW, Visual C⁺⁺, Visual Basic, MatLAB, etc., may be displayed on the display unit, e.g. a monitor, and stored into an auxiliary memory unit.

Biosignals such as heartbeat pulse wave and respiration pulse wave from the chest, head, wrist, ankle, etc., may be measured and displayed on the monitor on a real time basis and may be stored simultaneously into the auxiliary memory unit of peripheral equipments (e.g., PC, PDA or stand-alone device).

Signals from the sensing unit include any signals from physical changes other than heartbeat and respiration of a patient and, therefore, are additionally processed after passing through the analog signal processing unit. Mixture signals measured from the chest, abdomen, head, wrist and ankle, which include heartbeat and respiration signals, are displayed in real time waveforms on the monitor and stored into the auxiliary memory unit. Specifically, when the patient wears the elastic band-type apparatus for measuring respiratory rate or heart rate of the present invention, mixture signals of heartbeat and respiration waves are shown and, however, it is easy to discriminate between respiration waves and heartbeat waves since the amplitude of the heartbeat waves is 1/10 times that of the respiration waves and the frequency of the heartbeat waves is 3-6 times that of the respiration waves. Signals which are measured by wearing the elastic band-type apparatus for measuring respiratory rate or heart rate of the present invention on the head, wrist, ankle, etc. and without movement of the patient, associate only with heartbeat and, therefore, pulse wave velocity can be measured very easily.

The objective of the elastic band-type apparatus for measuring respiratory rate or heart rate of the present invention is to measure and analyze an accurate real time respiration and heartbeat pattern of a patient under general anesthesia as well as a mild patient and a normal person, and preceive an emergency of the patient such that a doctor, a nurse and a medical orderly can be immediately informed of the emergency. In addition, the measured signals may be digitalized and stored into various memory devices and, later, a doctor can trace the patient's condition back.

Moreover, these digital informations can be analyzed by using various programs (such as programs using LabVIEW, Visual C⁺⁺, Visual Basic, MatLAB, etc.) and immediately informed of nearby hospitals or public institutions by conventional communication systems.

### EXAMPLES

### Example 1

### Measurement of Respiration and Heartbeat Pulses

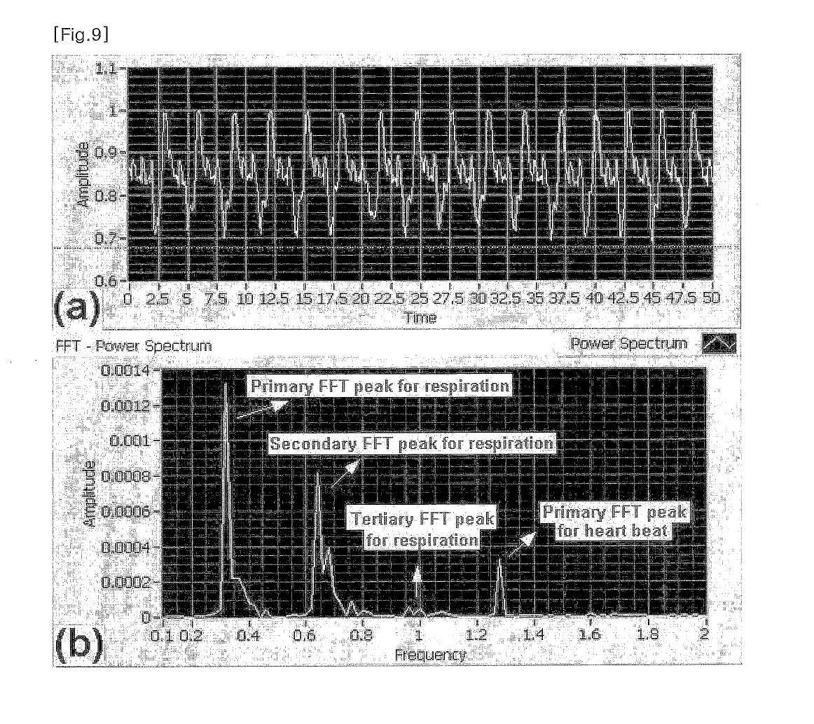

The present experiment was performed with wearing somewhat tightly the elastic band-type apparatus for measuring respiratory rate or heart rate of the present invention on the wrist, ankle, head, chest, etc. Pressures according to the amount of blood flow in blood vessels, are applied to the elastic band-type apparatus and, consequently, a sensor deforms. Deformation of the apparatus induces electrical signals from the sensor located within the apparatus, due to piezoelectricity. Thus induced signals are processed by the system for measuring respiratory rate or heart rate according to the present invention, which is represented in FIGS. 4-7, and then showed on a computer monitor as real-time waveforms. These waveforms are shown in FIG. 8(a) and FIG. 9(a).

Power spectra obtained through fast Fourier transform (FFT) of the frequency domain of the real-time waveforms shown in FIG. 8(a) and FIG. 9(a) are illustrated in FIG. 8(b) and FIG. 9(b).

It can be known that FIG. 8 shows only periodic waveforms associated with heartbeat since the elastic band-type apparatus was worn on the wrist. The primary peak of the power spectra appeared at 1.2 Hz after FFT of the periodic waveforms of FIG. 8(a) and, therefore, the heart rate is calculated as 1.2×60=72 min⁻¹.

FIG. 9 shows periodic respiration waveforms (with a larger amplitude) and heartbeat waveforms (with a smaller amplitude) simultaneously since the apparatus was worn on the chest. After FFT of the waveforms, the largest primary peak appeared at 0.32 Hz, the secondary peak of which amplitude was considerably decreased appeared at 0.64 Hz which is twice the frequency of the primary peak, and the tertiary peak of which amplitude was negligible compared to that of the primary peak appeared at 0.96 Hz which is three times the frequency of the primary peak. Therefore, the primary peak which appeared at 0.32 Hz is associated with respiration and, thus, the respiratory rate is calculated as 0.32×60=19.2 min⁻¹.

On the other hand, since the amplitude of a real-time heartbeat waveform is much smaller than that of a real-time respiration waveform and the frequency of a real-time heartbeat waveform is much larger than that of a real-time respiration waveform, the amplitude of the FFT peak for heartbeat is much smaller than the primary FFT peak for respiration and much larger than the tertiary FFT peak for respiration. Therefore, the FFT peak at 1.28 Hz is not the quaternary FFT peak for respiration but the primary FFT peak for heartbeat. Consequently, the heart rate is calculated as 1.28×60=76.8 min⁻¹.

### Example 2

Influence of Rubber Thickness Coated on Piezoelectric Film to Signals being Measured

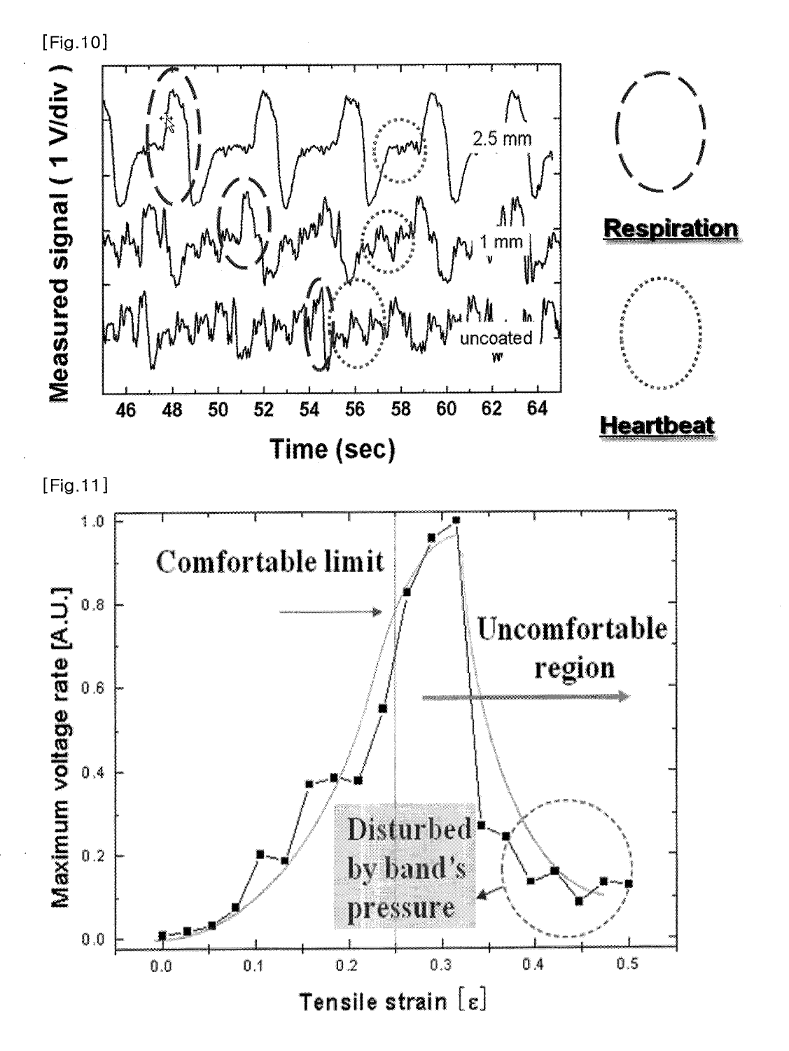

In order to find out the influence of rubber thickness coated on the piezoelectric film to signals being measured in response to respiration, three types of the elastic band-type apparatus, i.e. a piezoelectric film sensor without silicon rubber coating, a piezoelectric film sensor with 1 mm-thick silicon rubber coating, and a piezoelectric film sensor with 2.5 mm-thick silicon rubber coating, were tied around the chest respectively as the same manner as in Example 1, and then signals were measured in the same conditions in Example 1. The measured real-time respiration/heartbeat waveforms are shown in FIG. 10.

The real-time signals of respiration increased in proportion to the thickness of the silicon rubber coating. From these results, it can be understood that, as the thickness of rubber coating increases, the adhesion between the piezoelectric film and the elastic band improves and the slippage of the piezoelectric film, due to elongation of the elastic band, decreases and the piezoelectric film elongates accordingly.

### Example 3

### Influence of Tensile Strain of Elastic Band to Signals Being Measured

After the elastic band-type apparatuses with different tensile strain were worn on the wrists, amplitudes of pulse waves were measured as the same manner in Example 1, and then the amplitude of the pulse waves as a function of tensile strain the elastic band were represented in FIG. 11.

It can be observed that the signal intensity of the pulse waves increased abruptly as the tensile strain of the elastic band increased up to 0.3, and then the signal intensity of the pulse waves decreased suddenly as the tensile strain of the elastic band increased more than 0.3. Considering the signal intensity and comfortable wearing of the elastic band of the present invention for a long time, the tensile strain of 0.25 was found to be most appropriate.

When wearing the elastic band-type apparatus with high tensile strain, friction between the piezoelectric film and the elastic band increased and, therefore, the slippage of the piezoelectric film decreased such that circumference change of the body in response to respiration or heartbeat increased. Thus, the piezoelectric polymer film elongated well in response to the elongation of the elastic band and, therefore, signal intensity increased. However, for the case of too much high tensile strain, signal intensity decreased on the contrary since elongation of the elastic band in response to respiration and heartbeat decreased. Moreover, when the elastic band with very high tensile strain is worn on the wrist or ankle, the pressure induced by the very high tensile strain occlude arteries and, therefore, it may be difficult to measure pulse waves.

### Example 4

### Measurements of Multi-Channel Pulse Waveforms and Pulse Wave Velocity (PWV)

A multi-channel system was constructed, which is comprised of simultaneous wearing of the elastic band-type apparatus in Example 1 on the chest, head, right wrist and right ankle, and heartbeat waveforms were measured on the various body locations through a multi-channel analog-digital converter and a data collection device.

At the same time, measured data were displayed on a monitor on a real time basis, and pulse wave velocities at the body locations were calculated from the pulse time delay measurements and the pre-input location data of the elastic band. During the present experiment, breathing was stopped for 10 seconds in order to obtain only heartbeat waveforms at the chest.

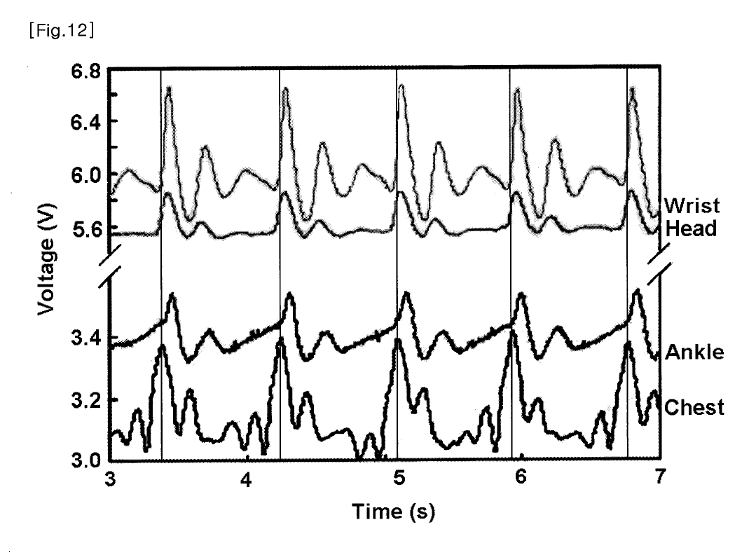

FIG. 12 shows pulse wave signals at four different body locations after wearing the elastic band-type apparatus of the present invention on the chest, head, right wrist and right ankle.

Referring to FIG. 12, it can be observed that the pulse wave delay increased in order of the head, wrist and ankle on the basis of the chest. Average pulse delay times of a normal person from the chest to the head, wrist and ankle were 73 ms, 119 ms and 148 ms, respectively, with standard deviation of less than or equal to 28 ms. As described above, health condition of arteries can be easily checked by measuring pulse delay times at various body locations.

### INDUSTRIAL APPLICABILITY

The sensor, apparatus and system of the present invention make it possible to measure length or circumference change of a part of an animal including a human, a plant, etc. Therefore, safety of a structure, growth of a plant, and respiratory rate or heart rate of an animal including a human can be measured.

The apparatus for measuring respiratory rate or heart rate of the present invention is advantageous to measure respiratory rate and heart rate since the apparatus does not give a patient uncomfortable feeling, can be easily wearable, and is very sensitive to physical change. Therefore, the apparatus can be used as means for measuring respiratory rate and heart rate of a patient in an emergency room, an operation room and an intensive care unit, etc. and also can be used for a patient monitoring system.

1. A sensor for measuring change of length or volume, which comprises a material which generates an electrical signal in response to elongation.

2. The sensor of claim 1, wherein said material is a piezoelectric polymer.

3. The sensor of claim 2, wherein said piezoelectric polymer is selected from the group consisting of poly(vinylidene fluoride), poly(vinylidene fluoride) blend, vinylidene fluoride copolymer and nylon-11.

4. The sensor of claim 3, wherein said poly(vinylidene fluoride) blend is selected from the group consisting of a poly(vinylidene fluoride)/poly(methyl methacrylate) blend, a poly(vinylidene fluoride)/poly(vinyl acetate) blend, and a poly(vinylidene fluoride)/poly(vinyl acetate) copolymer blend.

5. The sensor of claim 3, wherein said vinylidene fluoride copolymer is selected from the group consisting of poly(vinylidene fluoride-co-trifluoroethylene), poly(vinylidene fluoride-co-tetrafluoroethylene), poly(vinylidene fluoride-co-hexafluoroethylene) and poly(vinylidene fluoride-co-trichlorofluoroethylene).

6. The sensor of claim 2, wherein said piezoelectric polymer is in the form selected from the group consisting of a film, a sheet, a cylinder, a string, a strand, a fiber, a woven fabric and a nanofiber web.

7. An apparatus for measuring a length or volume change, which comprises a sensor comprising a material which generates an electrical signal in response to elongation, wherein said sensor is mounted on or around an object of measurement, and the length or volume change of the object is measured from an electrical signal in response to elongational deformation of the material due to the increase of length or circumference of the object.

8. The apparatus for measuring a length or volume change of claim 7, wherein said material is a piezoelectric polymer.

9. The apparatus for measuring a length or volume change of claim 8, wherein said piezoelectric polymer is selected from the group consisting of poly(vinylidene fluoride), poly(vinylidene fluoride) blend, vinylidene fluoride copolymer and nylon-11.

10. The apparatus for measuring a length or volume change of claim 9, wherein said poly(vinylidene fluoride) blend is selected from the group consisting of a poly(vinylidene fluoride)/poly(methyl methacrylate) blend, a poly(vinylidene fluoride)/poly(vinyl acetate) blend, and a poly(vinylidene fluoride)/poly(vinyl acetate) copolymer blend.

11. The apparatus for measuring a length or volume change of claim 9, wherein said vinylidene fluoride copolymer is selected from the group consisting of poly(vinylidene fluoride-co-trifluoroethylene), poly(vinylidene fluoride-co-tetrafluoroethylene), poly(vinylidene fluoride-co-hexafluoroethylene) and poly(vinylidene fluoride-co-trichlorofluoroethylene).

12. The apparatus for measuring a length or volume change of claim 8, wherein said piezoelectric polymer is in the form selected from the group consisting of a film, a sheet, a cylinder, a string, a strand, a fiber, a woven fabric and a nanofiber web.

13. An apparatus for measuring respiratory rate or heart rate, which comprises a sensor comprising a material which generates an electrical signal in response to elongation, wherein said sensor is mounted on or around a living being of measurement, and the respiratory rate or heart rate of the living being is measured from an electrical signal in response to elongational deformation of the material due to the increase of circumference of the living being.

14. The apparatus for measuring respiratory rate or heart rate of claim 13, wherein said material is a piezoelectric polymer.

15. The apparatus for measuring respiratory rate or heart rate of claim 14, wherein said piezoelectric polymer is selected from the group consisting of poly(vinylidene fluoride), poly(vinylidene fluoride) blend, vinylidene fluoride copolymer and nylon-11.

16. The apparatus for measuring respiratory rate or heart rate of claim 15, wherein said poly(vinylidene fluoride) blend is selected from the group consisting of a poly(vinylidene fluoride)/poly(methyl methacrylate) blend, a poly(vinylidene fluoride)/poly(vinyl acetate) blend, and a poly(vinylidene fluoride)/poly(vinyl acetate) copolymer blend.

17. The apparatus for measuring respiratory rate or heart rate of claim 15, wherein said vinylidene fluoride copolymer is selected from the group consisting of poly(vinylidene fluoride-co-trifluoroethylene), poly(vinylidene fluoride-co-tetrafluoroethylene), poly(vinylidene fluoride-co-hexafluoroethylene) and poly(vinylidene fluoride-co-trichlorofluoroethylene).

18. The apparatus for measuring respiratory rate or heart rate of claim 13, wherein said piezoelectric polymer is in the form selected from the group consisting of a film, a sheet, a cylinder, a string, a strand, a fiber, a woven fabric and a nanofiber web.

19. An elastic band-type apparatus for measuring respiratory rate or heart rate, which comprises a material layer which generates an electrical signal in response to elongation; two electrode layers, each of which covers respectively either side of said material layer; and an elastic band which wraps said material layer and said two electrode layers.

20. The elastic band-type apparatus for measuring respiratory rate or heart rate of claim 19, wherein said material is a piezoelectric polymer.

21. The elastic band-type apparatus for measuring respiratory rate or heart rate of claim 20, wherein said piezoelectric polymer is selected from the group consisting of poly(vinylidene fluoride), poly(vinylidene fluoride) blend, vinylidene fluoride copolymer and nylon-11.

22. The elastic band-type apparatus for measuring respiratory rate or heart rate of claim 21, wherein said poly(vinylidene fluoride) blend is selected from the group consisting of a poly(vinylidene fluoride)/poly(methyl methacrylate) blend, a poly(vinylidene fluoride)/poly(vinyl acetate) blend, and a poly(vinylidene fluoride)/poly(vinyl acetate) copolymer blend.

23. The elastic band-type apparatus for measuring respiratory rate or heart rate of claim 21, wherein said vinylidene fluoride copolymer is selected from the group consisting of poly(vinylidene fluoride-co-trifluoroethylene), poly(vinylidene fluoride-co-tetrafluoroethylene), poly(vinylidene fluoride-co-hexafluoroethylene) and poly(vinylidene fluoride-co-trichlorofluoroethylene).

24. The elastic band-type apparatus for measuring respiratory rate or heart rate of claim 19, wherein said piezoelectric polymer is in the form selected from the group consisting of a film, a rod, a woven fabric and a nanofiber web.

25. The elastic band-type apparatus for measuring respiratory rate or heart rate of claim 19, wherein said two electrode layers are selected from the group consisting of Au, Ag, Cu, Pt, Al, Ni and Co.

26. The elastic band-type apparatus for measuring respiratory rate or heart rate of claim 19, wherein said elastic band is produced from elastic fibers.

27. The elastic band-type apparatus for measuring respiratory rate or heart rate of claim 19, wherein at least one outer side of said two electrode layers is coated with a polymeric material.

28. The elastic band-type apparatus for measuring respiratory rate or heart rate of claim 27, wherein said polymeric material is at least one selected from the group consisting of butadiene rubber or latex, isoprene rubber or latex, chloroprene rubber or latex, nitrile rubber or latex, silicon rubber or latex, polyurethane rubber or latex, polyethylene, polyester, polyacryl, polyimide and polyacetate.

29. The elastic band-type apparatus for measuring respiratory rate or heart rate of claim 27, wherein the coating thickness of said polymeric material is between 100 μm and 5 mm.

30. The elastic band-type apparatus for measuring respiratory rate or heart rate of claim 19, wherein the tensile strain of said elastic band is between 0.1 and 0.4.

31. The elastic band-type apparatus for measuring respiratory rate or heart rate of claim 19, wherein conductive leads are connected to said two electrode layers.

32. A system for measuring respiratory rate or heart rate, which comprises:
a sensing unit comprising a sensor which generates an electrical signal in response to elongation;
an analog signal processing unit which removes noises from the analog signals measured from said sensor and amplifies noise-removed analog signals;
an analog-digital converting unit which converts said noise-removed analog signals into digital signals;
a digital signal processing unit which analyzes the digital signals from said analog-digital converting unit and thereby calculate a respiratory rate or heart rate; and
a display unit which displays the data of the respiratory rate or heart rate from said digital signal processing unit.

33. The system for measuring respiratory rate or heart rate of claim 32, further comprises an auxiliary memory unit.
